(19) United States
(12) Patent Application Publication
(10) Pub. No.: US 2012/0277193 A1
(43) Pub. Date: Nov. 1, 2012

(54) Synergistic Combination Therapy

(75) Inventors: Frederick P. Roth, Toronto (CA); Murat Cokol, Brookline, MA (US); Hon Nian Chua, Revere, MA (US)

(73) Assignee: PRESIDENT AND FELLOWS OF HARVARD COLLEGE, Cambridge, MA (US)

(21) Appl. No.: 13/497,272

(22) PCT Filed: Sep. 20, 2010
(86) PCT No.: PCT/US10/49453

§ 371 Date: Jul. 20, 2012

Related U.S. Application Data
(60) Provisional application No. 61/244,256, filed on Sep. 21, 2009.

(51) Int. Cl. A61K 31/436; A61K 31/675; A61K 31/4453; A61P 31/10; A61K 31/40; A61K 31/34; A61K 31/4184; A61P 31/04; A61K 31/5375; A61K 31/445

(52) U.S. Cl. 514/92; 514/291; 514/239.5; 514/317; 514/327; 514/425; 514/468; 514/395

(57) ABSTRACT

Combinations of compounds are provided that produce a synergistic effect when administered.

### RELATED APPLICATION

This application claims priority to U.S. Provisional Patent Application No. 61/244,256, filed on Sep. 21, 2009 and is hereby incorporated herein by reference in its entirety for all purposes.

### STATEMENT OF GOVERNMENT INTERESTS

This invention was made with Government support under Grant Number R01HG003224 awarded by the National Institutes of Health. The Government has certain rights in the invention.

### FIELD

The present invention relates to the combination of at least two compounds which provides a synergistic effect useful in the treatment of diseases and therapeutic methods using such combination. The present invention also includes kits containing the combination of compounds and methods of identifying compounds having a synergistic effect.

### BACKGROUND

Compounds are known that have certain therapeutic or other beneficial effects, for example in treating certain diseases of humans or plants or for use as fungicides, bactericides etc. However, the efficacy of the individual compound may not be sufficient to provide a reasonable treatment or other beneficial effect. In such cases, combination therapies can be used. See Auspitz US 2007/0110685, Bascomb US 2008/0033027, Borisy U.S. Pat. No. 6,846,816, and Johansen US 2006/0264384. Methods for identifying compounds that interact in a synergistic manner are therefore needed, as are the combinations of the compounds themselves, methods of using the compounds and kits including the compounds. Combinations may also achieve the desired efficacy with lower concentrations of each compound relative to the level that would be required to achieve the same efficacy with either individual compound. This can reduce undesired side effects.

### SUMMARY

Embodiments of the present invention are directed to compounds that have a synergistic effect. These compounds are administered in combination, which is understood to mean that the compounds are present in or on an organism at least for some common period of time. The effect of the individual compound may be therapeutic or otherwise. However, when the compound is combined with at least one other compound, i.e. its synergistic partner or pair, the resulting effect is increased relative to the effect of the compounds individually. According to one aspect of the present invention, the combination of compounds, or a pharmaceutically acceptable form thereof, is administered to an organism, including a human, animal, plant, fungus, bacteria etc. such that the effect of the compounds is increased compared to the effect of the compounds If administered individually. That is, the compounds produce a synergistic effect when administered or applied as a combination, thereby producing a more efficacious therapy or result. One example of the combinations of the present invention includes combinations of drugs or other therapeutic or agricultural agents that exhibit a synergistic effect when administered in combination. Embodiments of the present invention are also directed to methods of using the combination of compounds depending on the synergistic effect produced. As such, methods according to the present invention include therapeutic methods, diagnostic methods, prognostic methods, agricultural methods or other methods utilizing the particular synergistic effect of the combination of compounds.

According to embodiments of the present invention, two or more genes or gene products are identified that interact in a synergistic manner. For the two or more genes or gene products, an agent or compound that targets a first gene or gene product and the resulting effect is determined. This is referred to as an “agent-target.” A second or additional agent or compound that targets a second or additional gene or gene product and the resulting effect is also determined. For genes or gene products that exhibit a synergistic genetic interaction, the agents, e.g., compounds, that target a corresponding gene or gene target may exhibit a synergistic effect when administered in combination. Such a synergistic effect is determined by measuring the effect when the compounds are administered in combination and comparing the effect when the compounds are administered individually. According to this aspect of the pre§ent invention, a method is provided for selecting compounds to be administered in combination to produce a synergistic effect based on the identification of two or more genes or gene products that exhibit a synergistic genetic interaction.

According to further embodiments of the invention, agents or compounds exhibiting a synergistic effect when administered are provided in a kit. In one aspect, the kit includes the compounds forming the combination, a pharmaceutically acceptable carrier, and optionally, instructions for use. The compounds may be provided in separate formulations for administration separately for interaction within an organism or the compounds may be combined into a single formulation for administration. Alternatively, the kit may provide the compounds in a single formulation for administration. It is important to understand that the synergistic effect may not be dependent upon the manner in which the compounds are administered, so long as the compounds are able to act in the organism after administration to produce a synergistic effect.

It will be recognized by the person of ordinary skill in the art that the compounds, compositions, methods and kits disclosed herein provide significant advantages over prior technology. Compounds, compositions, methods and kits can be designed or selected to utilize beneficial synergistic effects, such as to treat disorders and/or to relieve and/or alleviate symptoms in a patient suffering from one or more disorders. These and other aspects and examples are described below.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1(a) depicts in graphical form an experimental setup for drug synergy testing.

FIG. 1(b) depicts a graph for assessing drug synergy.

FIG. 2 depicts experimental data demonstrating synergistic drug pairs staurosporine and tacrolimus; tacrolimus and latrunculin B; and tacrolimus and terbinafine.

FIG. 3 depicts experimental data demonstrating synergistic drug pairs tacrolimus and fenpropimorph; tacrolimus and haloperidol; and anisomycin and tacrolimus.

FIG. 4 depicts experimental data demonstrating synergistic drug pairs cantharidin and tacrolimus; cycloheximide and tacrolimus; and radicicol and tacrolimus.

FIG. 5 depicts experimental data demonstrating synergistic drug pairs staurosporine and terbinafine; staurosporine and dyclonine; and staurosporine and haloperidol.

FIG. 6 depicts experimental data demonstrating synergistic drug pairs latrunculin B and terbinafine; latrunculin B and fenpropimorph; and latrunculin B and dyclonine.

FIG. 7 depicts experimental data demonstrating synergistic drug pairs latrunculin B and haloperidol; rapamycin and terbinafine; and calyculin A and terbinafine.

FIG. 8 depicts experimental data demonstrating synergistic drug pairs terbinafine and fenpropimorph; terbinafine and dyclonine; and terbinafine and haloperidol.

FIG. 9 depicts experimental data demonstrating synergistic drug pairs 5-fluorouracil and terbinafine; anisomycin and terbinafine; and carbonyl cyanide 3-chlorophenylhydrazone and terbinafine.

FIG. 10 depicts experimental data demonstrating synergistic drug pairs cantharidin and terbinafine; radicicol and terbinafine; and rapamycin and fenpropimorph.

FIG. 11 depicts experimental data demonstrating synergistic drug pairs calyculin A and fenpropimorph; calyculin A and dyclonine; and calyculin A and haloperidol.

FIG. 12 depicts experimental data demonstrating synergistic drug pairs carbonyl cyanide 3-chlorophenylhydrazone and benomyl; carbonyl cyanide 3-chlorophenylhydrazone and pentamidine; and pentamidine and benomyl.

FIG. 13 depicts experimental data demonstrating synergistic drug pairs staurosporine and fenpropimorph; rapamycin and haloperidol; and lithium chloride and tacrolimus.

FIG. 14 depicts experimental data demonstrating synergistic drug pairs rapamycin and dyclonine.

It will be recognized that the results and examples in the figures are only illustrative and other examples and illustrations will be readily recognized by the person of ordinary skill in the art, given the benefit of this disclosure.

### DETAILED DESCRIPTION OF CERTAIN EMBODIMENTS

Embodiments of the present invention are directed to combinations of compounds that interact to produce a synergistic effect when administered to a subject in need of treatment or when contacted to a cell to achieve a desired result. The term “synergistic effect” is intended to have its ordinary meaning to one of skill in the art. A synergistic effect produced by a combination of compounds can include an effect which is observed to be greater than the effect produced by each compound individually. Preferably, there is at least one beneficial effect, for example an additive effect or a mutual enhancing of the effect of each individual compound by the combination of compounds and in particular a more than additive effect, additional advantageous effects, fewer or lessened side effects, or a combined therapeutic effect in a non-effective dosage of the individual compounds. Such effects include those useful for therapeutic or agricultural purposes and are determined by the interaction of the agent and the target gene. Useful effects include anticancer effects, antineoplastic effects, anti-proliferative effects, fungicidal effects, fungistatic effects, anti-immune effects, anti-inflammatory effects, anti-transplant rejection effects, cell death effects, bacteriocidal effects, effects on behavior or mood, effects on blood pressure, effects on satiety, effects leading to weight loss or gain, effects on blood pressure, effects on sensation of pain, antiviral effects, anti-protozoal effects, anti-infective effects. As used herein, an organism or subject is intended to include a cell, a plant, a human and non-human mammals. Examples of a non-human mammal include, but are not limited to, non-human primates, horses, cows, goats, sheep, dogs, cats, mice, rats, hamsters, guinea pigs and the like.

According to the present invention where synergistic effects result from administration of combinations of compounds of the present invention, lower doses of the active ingredients can be used because of the synergistic effect achieved. For example, dosages may be smaller and administered less frequently, or can be used to lessen side effects observed with one of the combination compounds alone. The synergistic effect produced by the combination is useful for eradication of pests, infections or tumors that are unresponsive or less responsive to administration of each individual compound alone.

One of skill in the art based on the disclosure herein can use established test models to demonstrate that the administered combination results in a synergistic effect. One of skill in the art can select a relevant in vitro test model or the pharmacological activity of the combination can be demonstrated in a clinical study. Suitable clinical studies are for example, open-label non-randomized, placebo-controlled, parallel studies in patients. Such studies are in particular suitable to compare the effects of administration of a single compound versus the effects of the administered combination.

According to the present invention, compounds of the present invention are selected from the following groups and are administered in combination to a subject in need of treatment or to a cell to achieve a desired result to produce a synergistic effect. Accordingly, any combination of compounds between groups and from within groups listed below are included within the scope of the present invention and without specifically listing all various combinations. One of skill in the art based on the present disclosure will readily be able to combine compounds from those listed below.

Compounds commonly referred to as non-steroidal immunophilin-dependent immunosuppressants (NSIDI) are useful compounds. Exemplary compounds include cyclosporine A, ABT-281, Isatx247, tacrolimus (FK506), ascomycin, pimecrolimus, rapamycin (sirolimus), everolimus, and the like.

Compounds commonly referred to as actin inhibitors are useful compounds. Exemplary compounds include latrunculin B, latrunculin A, cytochalasin, cytochalasin B, cytochalasin D, cytochalasin E, phalloidin and the like.

Compounds commonly referred to as allylamines and or squalene monooxygenase inhibitors are useful compounds. Exemplary compounds include terbinafine, amorolfine, butenafine and naftitine.

Compounds commonly referred to as ergosterol inhibitors are useful compounds. Exemplary compounds include terbinafine and the like. Further useful ergosterol inhibitors are those commonly referred to as azoles. Exemplary compounds include bifonazole, clomidazole, clotrimazole, croconazole, econazole, fenticonazole, ketoconazole, isoconazole, miconazole, neticonazole, oxiconazole, sertaconazole, sulconazole, tioconazole, fluconazole, fosfluconazole, itraconazole, posaconazole, voriconazole, thiabendazole and the like. Further useful ergosterol inhibitors include polyene antimycotics. Exemplary compounds include natamycin, nystatin, amphotericin B and the like.

Compounds commonly referred to as local anesthetics are useful compounds. Exemplary compounds include dyclonine, benzocaine, lidocaine, bupivacaine, cocaine and the like.

Compounds commonly referred to as antipsychotic butyrophenones are useful compounds. Exemplary compounds include haloperidol, droperidol, benperidol, triperidol, melperone, lenperone, azaperone, bromperidol, fluanisone, trifluperidol, domperidone and the like.

Compounds commonly referred to as morpholines are useful compounds. Exemplary compounds include fenpropimorph, fenpropidine, dodemorph, tridemorph and the like.

Compounds commonly referred to as antifungal alkaloids or antibiotics such as staurosporine-derived compounds, are useful compounds. Exemplary compounds include staurosporine, PKC412 (Midostaurin), K252C (Staurosporine aglycon) and the like.

Compounds commonly referred to as serine/threonine phosphatase inhibitors are useful compounds. Exemplary compounds include calyculin A and calyculin C.

Compounds commonly referred to as pyrimidine analogs, thymidylate synthase inhibitors or antimetabolites are useful compounds. Exemplary compounds include fluorouracil, floxuridine, flucytosine, gemcitabine, cytosine arabinoside and the like.

Compounds commonly referred to as protein synthesis inhibitors are useful compounds. Exemplary compounds include anisomycin, cycloheximide and the like.

Compounds that inhibit oxidative phosphorylation are useful compounds. Exemplary compounds include carbonyl cyanide 3-chlorophenylhydrazone and the like.

Compounds commonly referred to as terpenoids or isoprenoids are useful compounds. Exemplary compounds include cantharidin, isoprene, prenol, isovaleric acid, geranyl pyrophosphate, eucalyptol, limonene, pinene, farnesyl pyrophosphate, artemisinin, bisabolol, retinol, phytol, taxol, forskolin, aphidicolin, squalene, lanosterol, lycopene, carotene and the like.

Compounds commonly referred to as heat shock protein inhibitors are useful compounds. Exemplary compounds that are known to inhibit heat shock proteins (Hsp90) in yeast include radicicol, prednisolone, ansamycin, geldanamycin and the like.

Compounds commonly referred to as benzimidazoles are useful compounds. An exemplary compound, benomyl, is currenly used as a fungicide due to its selective toxicity against microorganisms and invertebrates.

Pentamidine is a useful compound which is currently used for treatment of pneumonia and trypanosome.

Carbonyl cyanide 3-chlorophenylhydrazone (CCCP) is a useful chemical which inhibits oxidative phosphorylation. Exemplary chemicals that inhibit oxidative phosphorylation include oligomycin, 2,4-dinitrophenol and rotenone.

Agents that kill or inhibit fungal cell growth, such as miconazole and fluconazole, are a generally useful class of agents for therapeutic or agricultural purposes. Terbinafine, staurosporine and fenpropimorph are known to be useful as either therapeutic or agricultural agents. Specifically, terbinafine is used to treat Athlete's foot and other skin related fungal infections, see A. K. Gupta, E. A. Cooper, Mycopathologia 166, 353 (2008) incorporated herein by reference in its entirety.

FK506 is known for its antifungal activity, see J. R. Blankenship, W. J. Steinbach, J. R. Perfect, J. Heitman, Curr. Opin. Investig Drugs 4, 192 (2003) incorporated herein by reference in its entirety, but is used as an immunosuppressive drug. One of its notable side-effects is yeast infection. According to the present invention and to the object of using synergistic combinations to reduce side effects, staurosporine, latrunculin B, anisomycin, cantharidin, cycloheximide, radicicol or lithium administered in conjunction with FK506 is a method to counteract this side effect of FK506, or the combinations are envisioned to specifically to treat yeast infection.

Morpholines are used as an emulsifier in the process of waxing fruit, which protects fruits against insect and fungal contamination. According to the present invention, latrunculin B, staurosporine, FK506, rapamycin, calyculin A, terbinafine or derivatives are synergistic with fenpropimorph and derivatives. This combination can therefore be used to specifically protect fruits against insects or fungi when employed in the waxing process.

Morpholines are used in cereals as antifungals after a resistance against demethylation inhibitors (miconazole, clotrimazole, triarimol) was developed by the fungal agents. According to the present invention, latrunculin B, staurosporine, FK506, rapamycin, calyculin A, terbinafine or derivatives are synergistic with fenpropimorph and derivatives. This combination can therefore be used to specifically protect cereals against fungal growth.

According to the present invention, two of the above compounds can be combined into a synergistic pair. According to an additional aspect of the present invention at least two or more of the above compounds produce a synergistic effect when administered as a combination. Accordingly, a useful combination can include at least 2, 3, 4, 5, 6, 7, 8, 9, 10 or more of the above compounds. One of skill in the art will readily understand the known therapeutic and mechanistic uses for each of the above compounds without specifically identifying each use herein. Preferably, a compound is selected from each of two or more of the above groups and the compounds are administered as a combination to produce a synergistic effect. Embodiments of the present invention include two or more compounds from a single group in combination with one or more compounds from an additional group or groups. According to the present invention, the following preferred combinations of compounds produce a synergistic effect: staurosporine & tacrolimus (FK506); latrunculin B & tacrolimus (FK506); terbinafine & tacrolimus (FK506); fenpropimorph & tacrolimus (FK506); haloperidol & tacrolimus (FK506); tacrolimus (FK506) and anisomycin; tacrolimus (FK506) & cantharidin; tacrolimus (FK506) & cycloheximide; tacrolimus (FK506) & radicicol; terbinafine & staurosporine; dyclonine & staurosporine; haloperidol & staurosporine; terbinafine & latrunculin B; fenpropimorph & latrunculin B; dyclonine & latrunculin B; haloperidol & latrunculin B; terbinafine & rapamycin; terbinafine & calyculin A; fenpropimorph & terbinafine; dyclonine & terbinafine; haloperidol & terbinafine; terbinafine & 5-fluorouracil; terbinafine & anisomycin; terbinafine & carbonyl cyanide 3-chlorophenylhydrazone; terbinafine & cantharidin; terbinafine & radicicol; fenpropimorph & rapamycin; fenpropimorph & calyculin A; dyclonine & calyculin A; haloperidol & calyculin A; benomyl & carbonyl cyanide 3-chlorophenylhydrazone; pentamidine & carbonyl cyanide 3-chlorophenylhydrazone; benomyl & pentamidine; fenpropimorph & staurosporine; haloperidol & rapamycin; tacrolimus & lithium; dyclonine & rapamycin and the like.

Combination therapy or co-therapy includes the administration of at least two of the compounds of the present invention to provide the effect from the co-action of the compounds. The effect of the combination includes, but is not limited to, pharmacokinetic or pharmacodynamic co-action resulting from the combination of the compounds. Administration of the compounds in combination is typically carried out over a defined time period (usually minutes, hours, days or weeks depending upon the combination selected). Combination therapy may encompass the administration of two or more of the compounds of the present invention as part of separate monotherapy regimens that result in the combinations of the present invention. Combination therapy is intended to embrace administration of the compounds of the present invention in a sequential manner, that is, wherein each compound is administered at a different time, as well as administration of the compounds in a substantially simultaneous manner. For example, two or more compounds can be administered within 10 days of each other, five days of each other, within 24 hours of each other, hours of each other, minutes of each other, seconds of each other or even simultaneously. Substantially simultaneous administration can be accomplished, for example, by administering to the subject a single capsule having a fixed ratio of the compounds or in multiple, single capsules for each of the compounds. Sequential or substantially simultaneous administration of each compound can be effected by any appropriate route including, but not limited to, inhalation, oral routes, intravenous routes, intramuscular routes, subcutaneous, rectal, intraperitoneal, parenteral, direct application, topical administration, transdermal, gastrointestinal, and direct absorption through mucuous membrane tissues. The compounds can be administered by the same or different routes and in the same or different sequence. For example, a first compound can be administered by intravenous injection while the other therapeutic agents or agents of the combination may be administered orally or vice versa.

Compounds of the present invention include analogs and derivatives thereof, as well as pharmaceutically acceptable salts, isomers, tautomers, metabolites, and the like. Pharmaceutically acceptable salts include acid addition salts and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium or ferric hydroxides, and such organic bases as isopropylamine, trimethylamine, histidine, procaine, and the like. Isomers include conformational isomers, steroisomers, geometric isomers, enantiomers and the like which exhibit biological or pharmacological activity.

In accordance with certain other examples, kits for treating one or more conditions are provided. In one example, the kit may comprise two or more of the compounds of the present invention. In another example, the kit may comprise a pharmaceutically acceptable carrier. In an additional example, the kit may also include instructions for treating one or more conditions.

In accordance with certain examples, the compounds of the present invention can be incorporated into pharmaceutical compositions suitable for administration. Such compositions typically comprise the compounds disclosed here and a pharmaceutically acceptable carrier. As used herein the term “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In accordance with certain examples, a pharmaceutical composition of the invention is formulated to be compatible with its intended route of administration. Such pharmaceutical compositions may be administered by inhalation, transdermally, orally, rectally, transmucosally, intestinally, parenterally, intramuscularly, subcutaneously, intravenously or other suitable methods that will be readily selected by the person of ordinary skill in the art, given the benefit of this disclosure. For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampules, disposable syringes or multiple dose vials made of glass or plastic.

In accordance with other examples, pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, CREMPHOR EL™ (BASF, Parsippany, N.J.), or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

In accordance with other examples, sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation can be vacuum drying and freeze-drying which yields a powder of the active ingredient plus any additional desired ingredient from a previously sterile-filtered solution thereof. Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

In at least certain examples, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These may be prepared according to methods known to those skilled in the art, for example, as described in U.S. Pat. No. 4,522,811, incorporated herein by reference in its entirety for all purposes.

In accordance with certain examples, pharmaceutical compositions of the invention comprise one or more compounds of the present invention covalently linked to a peptide (i.e., a polypeptide comprising two or more amino acids). Peptides may be assembled sequentially from individual amino acids or by linking suitable small peptide fragments. In sequential assembly, the peptide chain is extended stepwise, starting at the C-terminus, by one amino acid per step. In fragment coupling, fragments of different lengths can be linked together, and the fragments can also be obtained by sequential assembly from amino acids or by fragment coupling of still shorter peptides.

In both sequential assembly and fragment coupling it is necessary to link the units (e.g., amino acids, peptides, compounds and the like) by forming an amide linkage, which can be accomplished via a variety of enzymatic and chemical methods. The methods described herein for formation of peptidic amide linkages are also suitable for the formation of non-peptidic amide linkages.

Chemical methods for forming the amide linkage are described in detail in standard references on peptide chemistry, including Muller, Methoden der organischen Chemie Vol. XV/2, 1-364, Thieme Verlag, Stuttgart, (1974); Stewart and Young, Solid Phase Peptide Synthesis, 31-34 and 71-82, Pierce Chemical Company, Rockford, Ill. (1984); Bodanszky et al., Peptide Synthesis, 85-128, John Wiley & Sons, New York, (1976); Practice of Peptide Synthesis, M. Bodansky, A. Bodansky, Springer-Verlag, 1994 and other standard works in peptide chemistry, incorporated herein by reference in their entirety for all purposes. Methods include the azide method, the symmetric and mixed anhydride method, the use of in situ generated or preformed active esters, the use of urethane protected N-carboxy anhydrides of amino acids and the formation of the amide linkage using coupling reagents, such as dicyclohexylcarbodiimide (DCC), diisopropylcarbodiimide (DIC), 1-ethoxycarbonyl-2-ethoxy-1,2-dihydroquinoline (EEDQ), pivaloyl chloride, 1-ethyl-3-(3-dimethylaminopropyl)carbodiimide hydrochloride (EDCI), n-propane-phosphonic anhydride (PPA), N,N-bis(2-oxo-3-oxazolidinyl)amido phosphoryl chloride (BOP-Cl), bromo-tris-pyrrolidinophosphonium hexafluorophosphate (PyBrop), diphenylphosphoryl azide (DPPA), Castro's reagent (BOP, PyBop), O-benzotriazolyl-N,N,N,N′-tetramethyluronium salts (HBTU), O-azabenzotriazolyl-N,N,N′,N′-tetramethyluronuim salts (TATU), diethylphosphoryl cyanide (DEPCN), 2,5-diphenyl-2,3-dihydro-3-oxo-4-hydroxythiophene dioxide (Steglich's reagent; HOTDO), 1,1′-carbonyldiimidazole (CDI) and the like. The coupling reagents can be employed alone or in combination with additives such as N,N-dimethyl-4-aminopyridine (DMAP), N-hydroxy-benzotriazole (HOBt), N-hydroxybenzotriazine (HOOBt), N-hydroxysuccinimide (HOSu), 2-hydroxypyridine and the like.

One embodiment of the present invention involves a method of treating a condition that includes the step of administering a therapeutically and/or prophylactically effective amount of each of at least two or more compounds of the present invention to a subject. As defined herein, a therapeutically and/or prophylactically effective amount of agent (i.e., an effective dosage) ranges from about 0.001 to 300 mg/kg body weight, from about 0.001 to 100 mg/kg body weight, from about 1 to 75 mg/kg body weight, from about 0.01 to 25 mg/kg body weight, from about 0.1 to 20 mg/kg body weight, from about 1 to 10 mg/kg, from about 2 to 9 mg/kg, from about 3 to 8 mg/kg, from about 4 to 7 mg/kg, or from about 5 to 6 mg/kg body weight. The skilled artisan will appreciate that certain factors may influence the dosage required to effectively treat a subject, including but not limited to the severity of the disease or disorder, the particular combination of compounds, mode of administration, previous treatments, the general health and/or age of the subject, and other diseases present. Treatment of a subject with a therapeutically and/or prophylactically effective amount of a combination of compounds can include a single treatment or can include a series of treatments. It will also be appreciated that the effective dosage for treatment may increase or decrease over the course of a particular treatment.

### Example I

### General Approach to Selecting Synergistic Compounds

According to aspects of the present invention, a method is provided for selecting compounds that are synergistic pairs. One of skill in the art will readily understand the term synergistic effect. However, solely for purposes of this disclosure and without intending to limit the invention in any way, two compounds or agents were said to exhibit a synergistic effect where the two compounds or agents X and Y present at concentrations C_{X} and C_{Y} exhibited a combined effect more extreme than the maximum effect attained when administering either X alone at a concentration 2C_{X} or Y alone at a concentration 2C_{Y}. See W. R. Greco, G. Bravo, J. C. Parsons, Pharmacol. Rev. 47, 331 (1995) incorporated herein by reference in its entirety. Solely for purposes of this disclosure and without intending to limit the invention in any way, two genes have a synergistic genetic interaction if the deletion of both genes results in a surprisingly slow growth given the growth rate of each single deletion. See R. Mani, R. P. St Onge, J. L. Hartman 4th, G. Giaever, F. P. Roth, Proc. Natl. Acad. Sci. U.S.A. 105, 3461 (2008) incorporated herein by reference in its entirety.

According to aspects of the present invention, if an agent inhibits the product of a gene, then the effect of the agent on the organism is predicted to be similar to the effect of a hypomorphic or null mutation in that gene. Since many agents work by inhibiting their targets, two agents whose targets have a synergistic genetic interaction are more likely to have a synergistic effect. Accordingly, one aspect of the present invention is to identify compounds that are synergistic pairs if they inhibit genes or products of genes that likewise exhibit a synergistic genetic interaction. Aspects of the present invention also are directed to identifying antagonistic agent pairs. See M. Hegreness, N. Shoresh, D. Damian, D. Hartl, R. Kishony, Proc. Natl. Acad. Sci. U.S. A. 105, 13977 (2008) incorporated herein by reference in its entirety. This aspect of the present invention provides advantages beyond current alternative methods for systematic discovery of agent synergies using simple experimental testing where success rate is very low. See A. A. Borisy et al., Proc. Natl. Acad. Sci. U.S.A. 100, 7977 (2003) incorporated herein by reference in its entirety. Moreover, experimental search is costly, since it requires large quantities of chemicals or other agents which are often expensive. Experimental search is slow as each agent pair must be tested for synergy in a ‘concentration matrix’ analysis, which requires growing cells of different combinations of concentrations for each agent, and assessing the growth to conclude synergy.

According to aspects of the present invention, genetic interactions can be measured in multiple model systems such as S. cerevisiae, E. coli, C. elegans, Drosophila, mouse, human cell culture, etc. (see N. J. Brideau et al., Science 314, 1292 (2006); G. Butland et al., Nat. Methods 5, 789 (2008); G. D. Gale et al., Mol. Psychiatry. 14, 631 (2009); B. Lehner, C. Crombie, J. Tischler, A. Fortunato, A. G. Fraser, Nat. Genet. 38, 896 (2006) incorporated herein by reference in their entirety), so that the present invention may be applied in the model system most relevant to the desired therapeutic or agricultural application for determining synergistic genetic interactions. One of skill in the art will understand that particular genetic interactions may be strain, environment or organism dependent and will adjust models for determining genetic interactions accordingly to take into account such variability using methods known to those of skill in the art. One of skill in the art will also understand that some agents may have desired effects that are achieved (in whole or part) through action on secondary targets for which genetic interaction has not been assessed, and so the effect of a single agent may not be similar to the effect of a hypomorphic or null allele of its target gene. However, one of skill in the art will also adjust models for determining genetic interactions accordingly to take into account such variability using methods known to those of skill in the art.

Using appropriate models to determine genetic interactions is advantageous insofar as the cost of identifying genetic interactions should be cheaper than experimental search for agent synergies. In terms of net cost, agent synergy testing requires often expensive agents used in combination at various concentrations with appropriate controls. Genetic interaction requires the appropriate genetically mutant strain and appropriate controls, but no agent. Testing of genetic interaction can be less expensive, especially where the mutant organisms are already available and the testing is done systematically. While the experimental time to test a single pair of potentially synergistic agents each at a given concentration is comparable to testing a single gene pair for synergistic genetic interaction, according to the present invention, high-throughput technologies for determining genetic interactions can generate thousands of genetic interactions. Genetic interactions are being systematically collected in multiple genetic organisms using a variety of high-throughput methods. See A. H. Tong et al., Science 303, 808 (2004); M. Schuldiner et al., Cell 123, 307 (2005); L. Decourty et al., Proc. Natl. Acad. Sci. U.S.A. 105, 5821 (2008); X. Pan et al., Cell 124, 1069 (2006); A. Friedman, N. Perrimon, Nature 444, 230 (2006) incorporated herein by reference in their entirety.

### Example II

### Identification of Synergistic Compounds

According to aspects of the present invention, a synergistic genetic interaction between the corresponding genes ACT1 and CNB1 was determined using a S. cerevisiae genetic interaction dataset provided by the BioGRID database and a genetic interaction dataset available from the University of Toronto created by Charles Boone and Brenda Andrews. Drugs that interacted with the genes were then identified. Latrunculin B is an inhibitor of the ACT1 gene product and FK506 is an inhibitor of the CNB1 gene product. Based upon the synergistic genetic interaction between the targets ACT1 and CNB1 of the two drugs Latrunculin B and FK506, the combination of Latrunculin B and FK506 was identified as producing a synergistic effect when administered in combination. Experimental results confirmed the synergistic effect compared to the effect each agent had when administered as a single agent at a selected concentration.

According to aspects of the present invention the method for identifying compounds having a synergistic effect can be extended to sets of more than two agents. Genetic interaction can be determined for sets of 3 or more genes, and where the corresponding gene set exhibits a synergistic genetic interaction, the agents interacting with the genes or gene products can be selected as having a synergistic effect when administered as a combination.

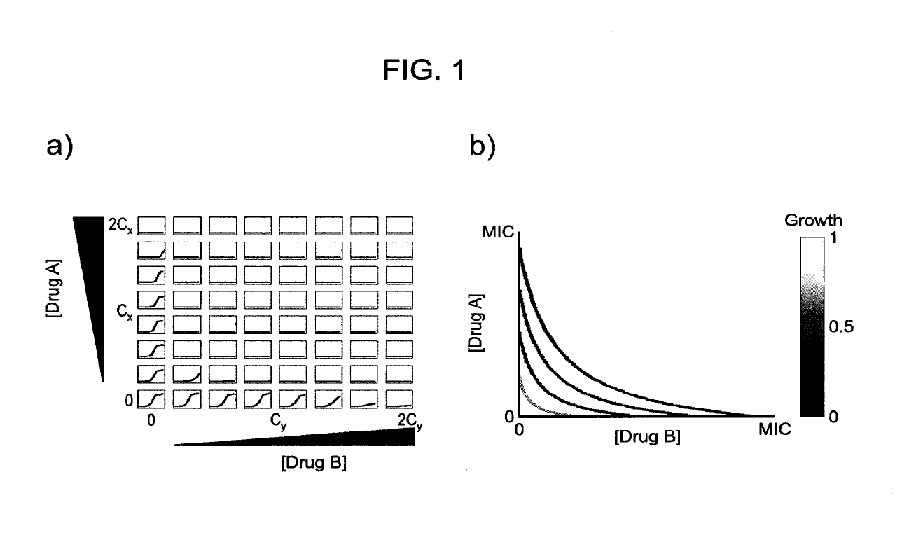

To experimentally verify drug synergies, a minimum inhibitory concentration (MIC) for each drug was identified. A ‘concentration matrix’ experiment was then performed in which drug combinations (C_{X}, C_{Y}) were produced such that the two drugs are present at concentrations C_{X} and C_{Y}, respectively. All 64 combinations were produced such that each drug was at one of eight concentrations at regular intervals between 0 and the MIC for that drug (See FIG. 1a). If, for any drug combination (C_{X}, C_{Y}), the effect on growth was more than was observed for 2C_{X} of drug X alone or with 2C_{Y} of drug Y alone, then the drugs were considered as producing a synergistic effect. FIG. 1b shows ‘isophenotypic’ curves, such that each combination (C_{X}, C_{Y}) along a given curve achieves the same specific phenotypic effect. A departure from the diagonal in a concave way as shown indicates synergy between drug X and drug Y.

According to the methods of the present invention, the following drug combinations were determined from synergistic gene interaction and verified through experiment to act synergistically to reduce the growth rate of S. cerevisiae: staurosporine and tacrolimus (FK506); latrunculin B and tacrolimus (FK506); latrunculin B and terbinafine; terbinafine and rapamycin; fenpropimorph and latrunculin B; dyclonine and latrunculin B; haloperidol and latrunculin B; and tacrolimus and lithium.

According to the methods of the present invention, the following drug combinations were experimentally shown to be acting synergistically to reduce the growth rate of S.cerevisiae: terbinafine and tacrolimus (FK506); fenpropimorph and tacrolimus (FK506); haloperidol and tacrolimus (FK506); tacrolimus (FK506) and anisomycin; tacrolimus (FK506) and cantharidin; tacrolimus (FK506) and cycloheximide; tacrolimus (FK506) and radicicol; terbinafine and staurosporine; dyclonine and staurosporine; haloperidol and staurosporine; terbinafine and calyculin A; fenpropimorph and terbinafine; dyclonine and terbinafine; haloperidol and terbinafine; terbinafine and 5-fluorouracil; terbinafine and anisomycin; terbinafine and carbonyl cyanide 3-chlorophenylhydrazone; terbinafine and cantharidin; terbinafine and radicicol; fenpropimorph and rapamycin; fenpropimorph and calyculin A; dyclonine and calyculin A; haloperidol and calyculin A; benomyl and carbonyl cyanide 3-chlorophenylhydrazone; pentamidine and carbonyl cyanide 3-chlorophenylhydrazone; benomyl and pentamidine; fenpropimorph and staurosporine; haloperidol and rapamycin; dyclonine and rapamycin.

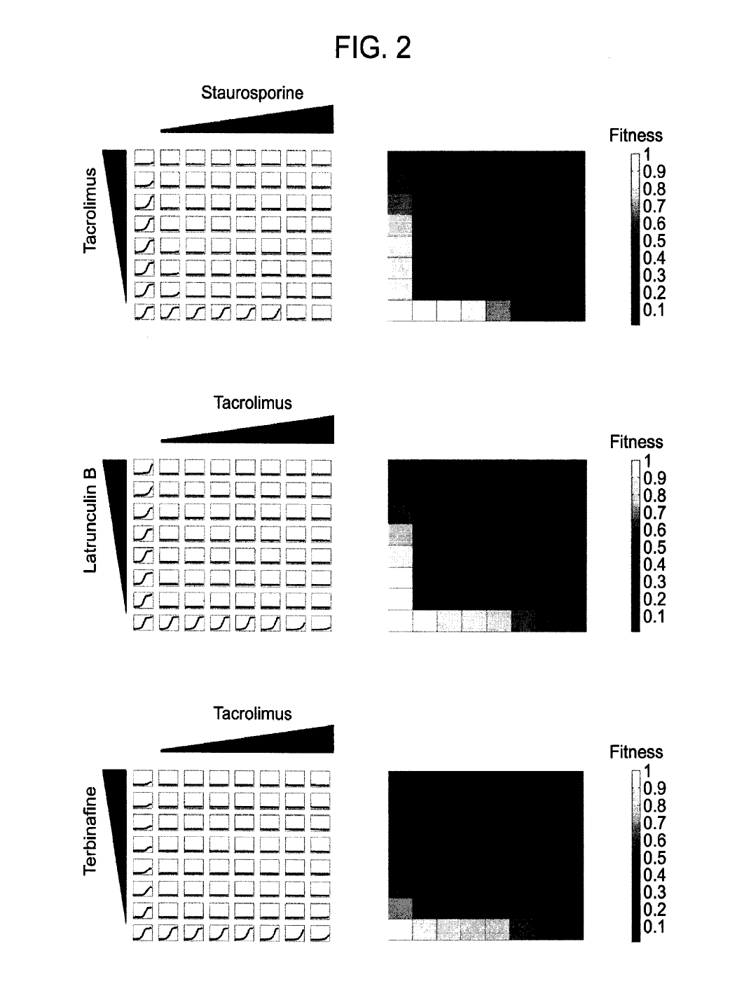

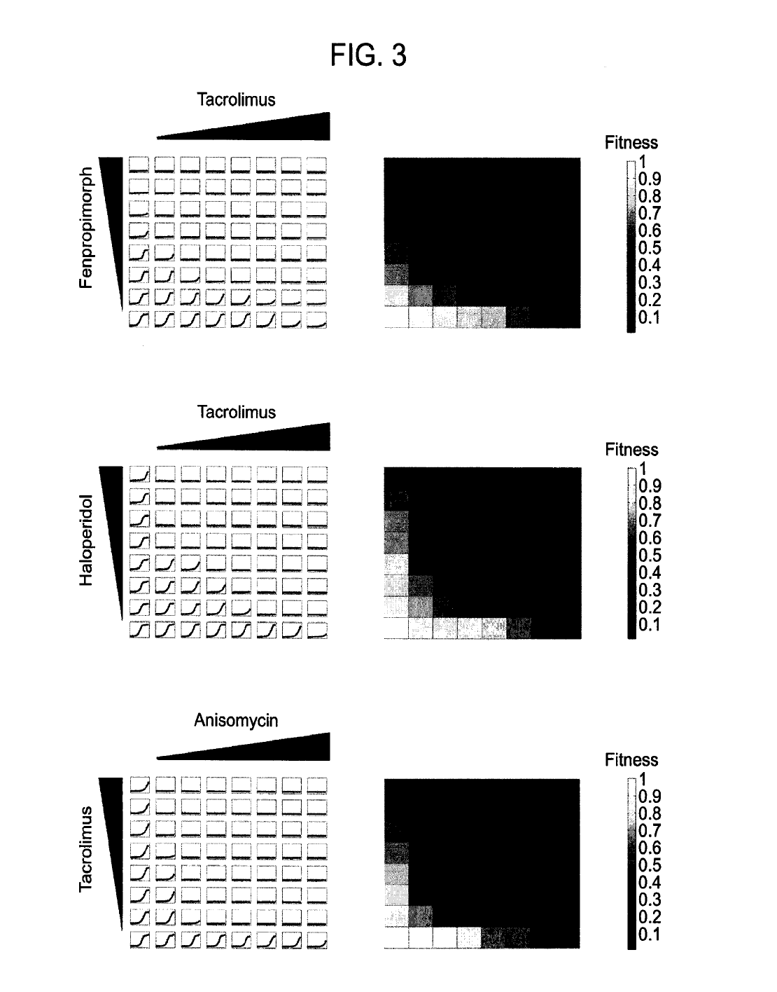

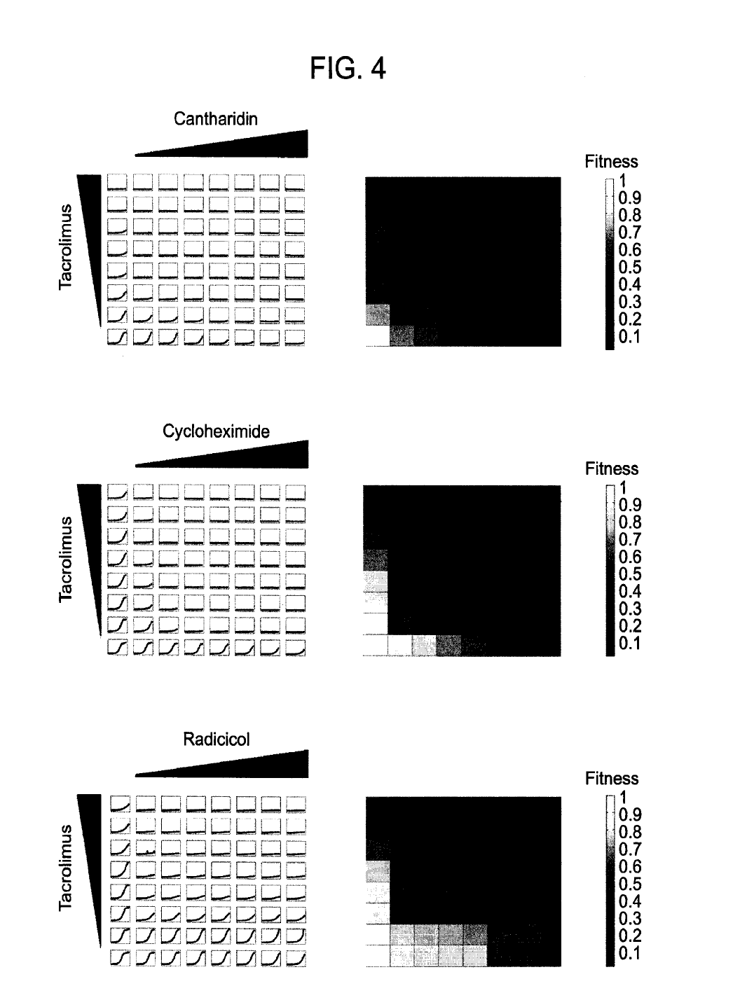

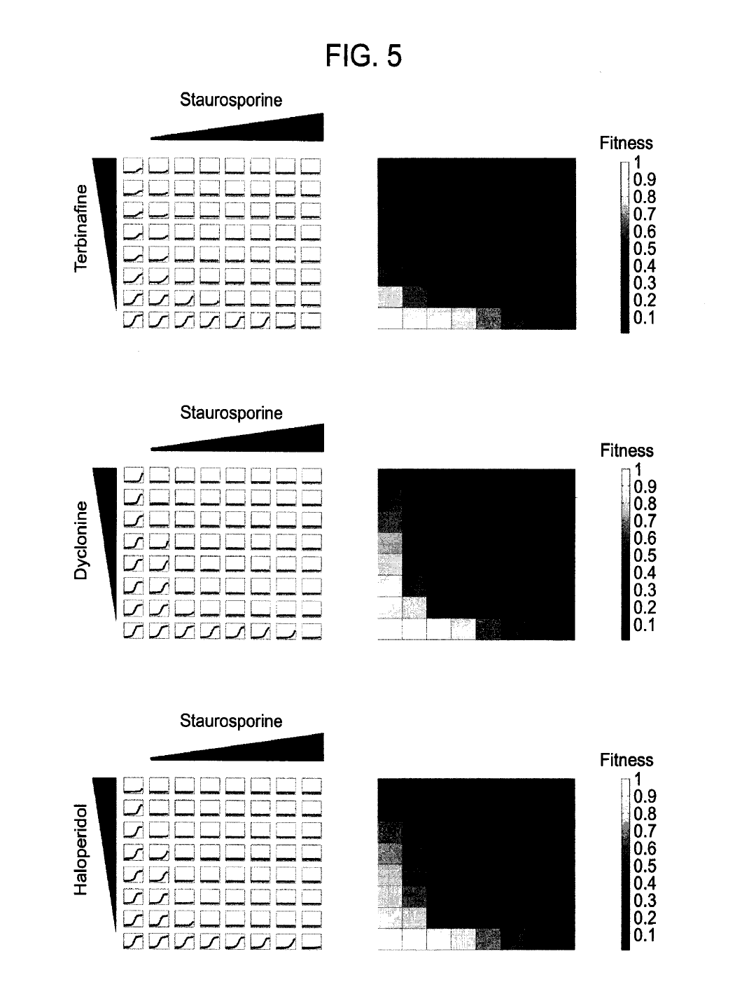

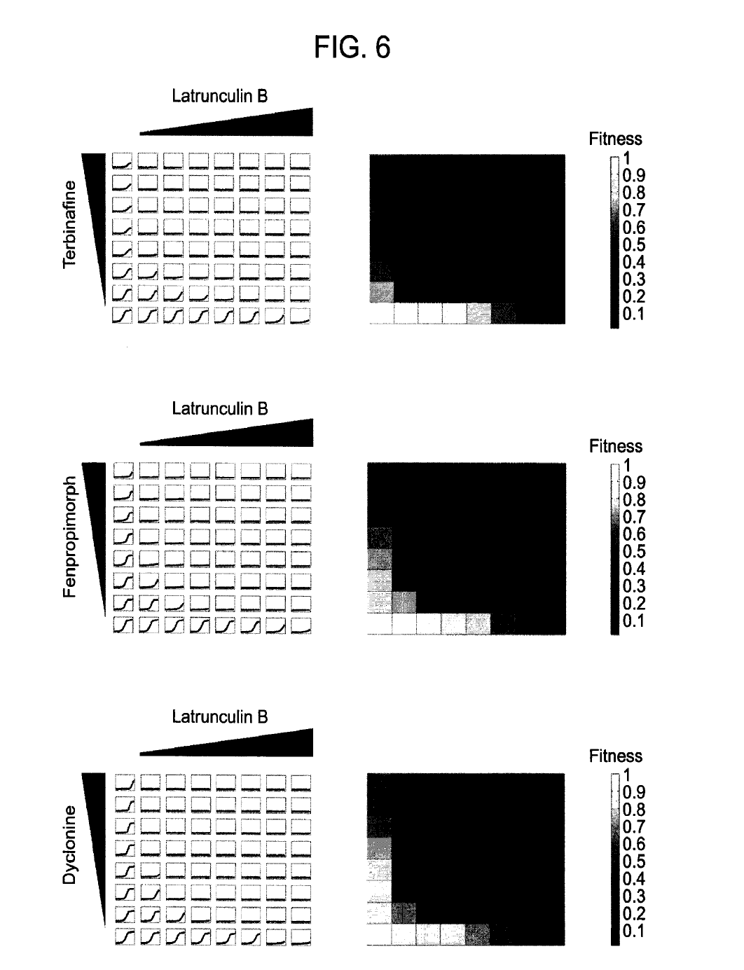

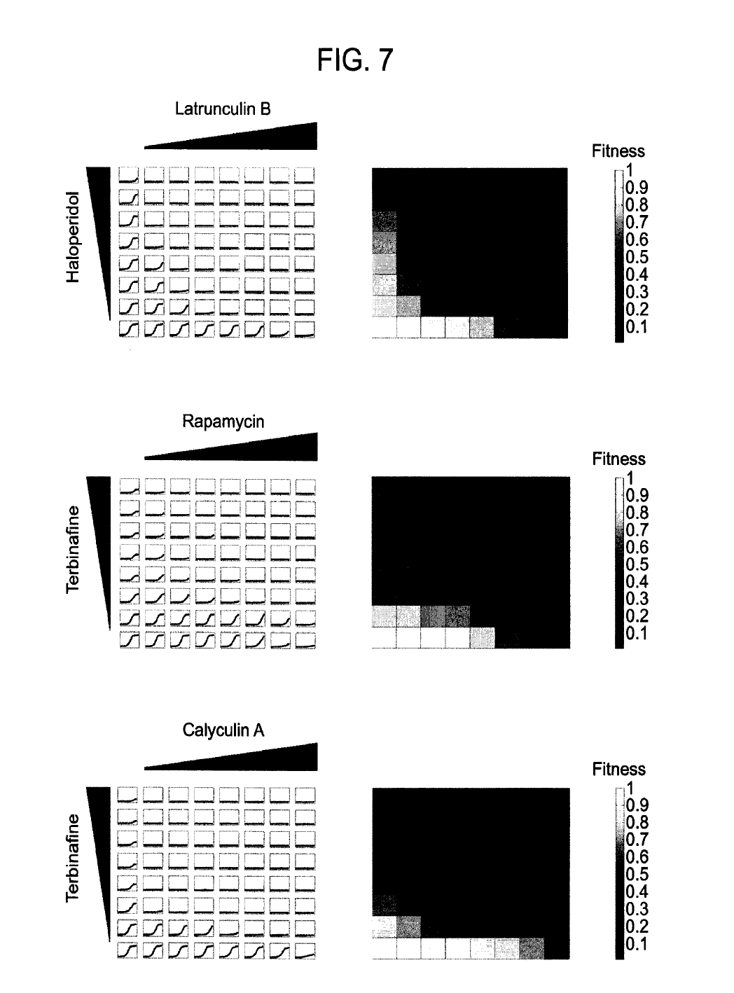

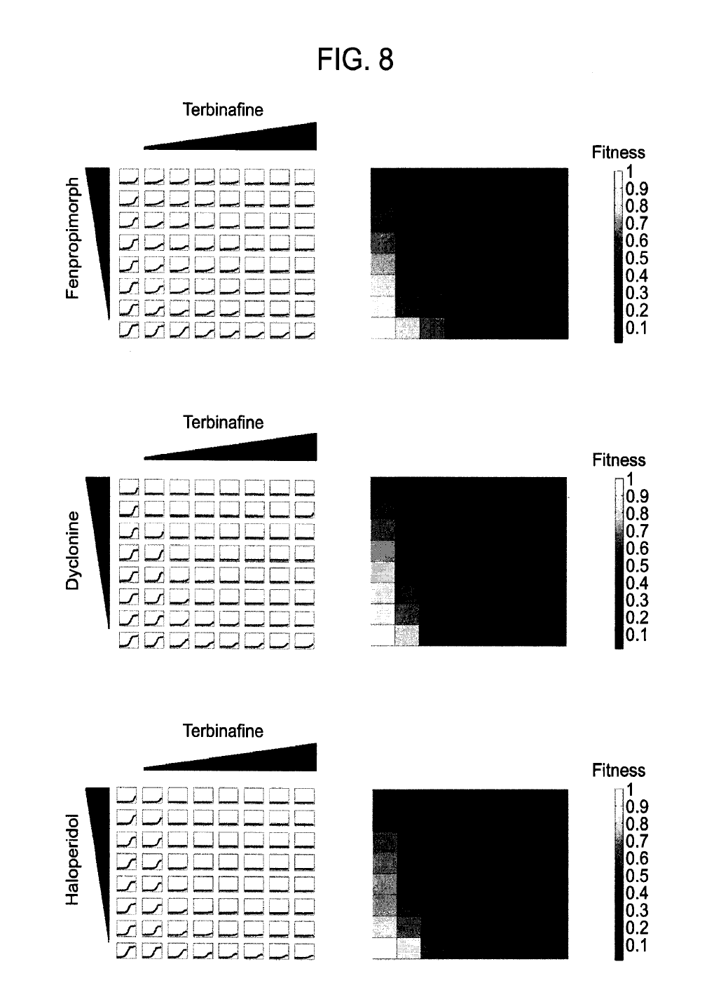

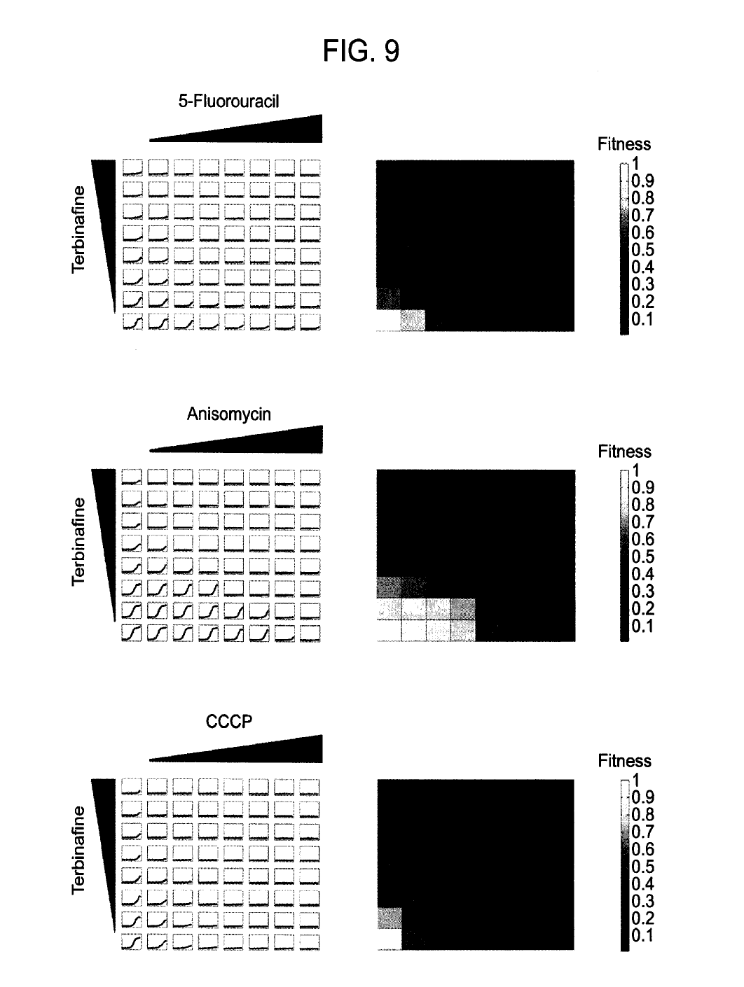

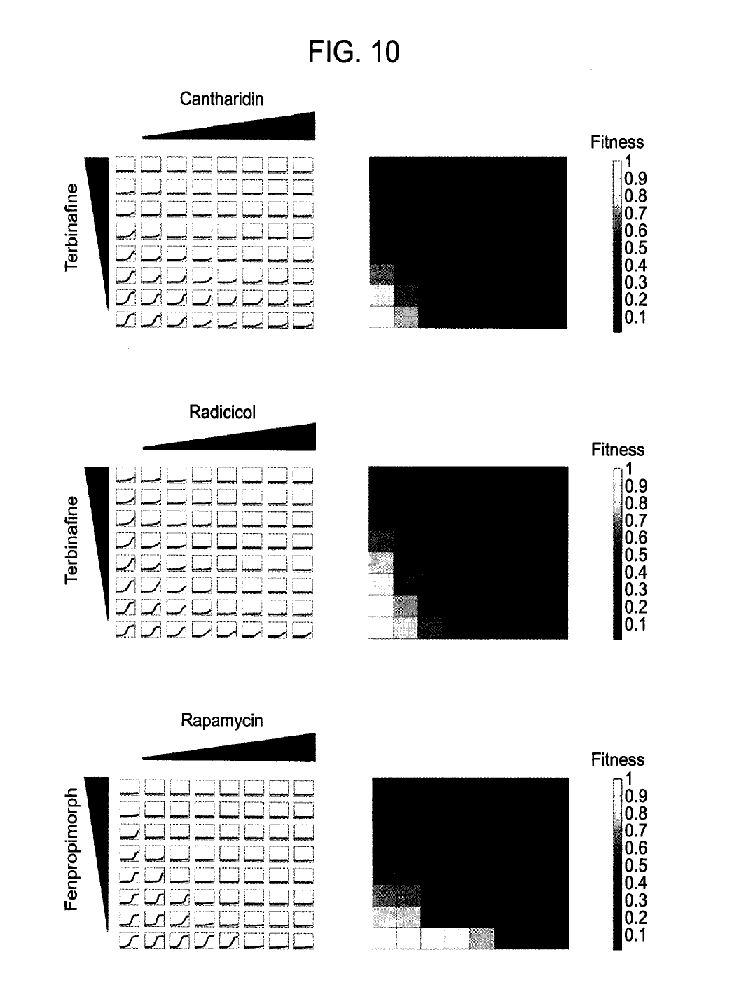

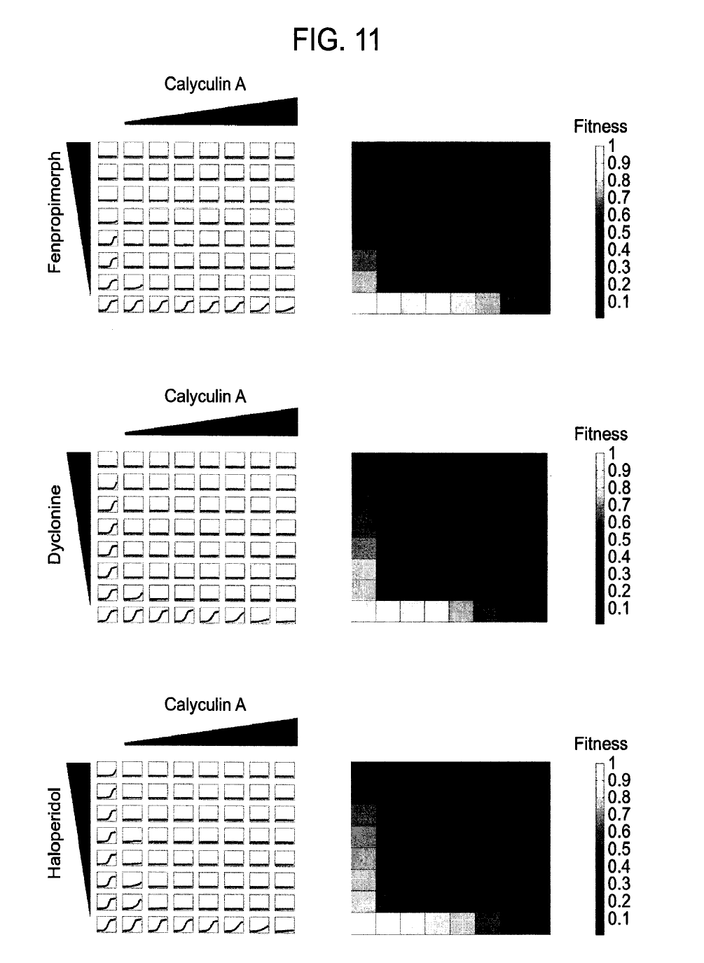

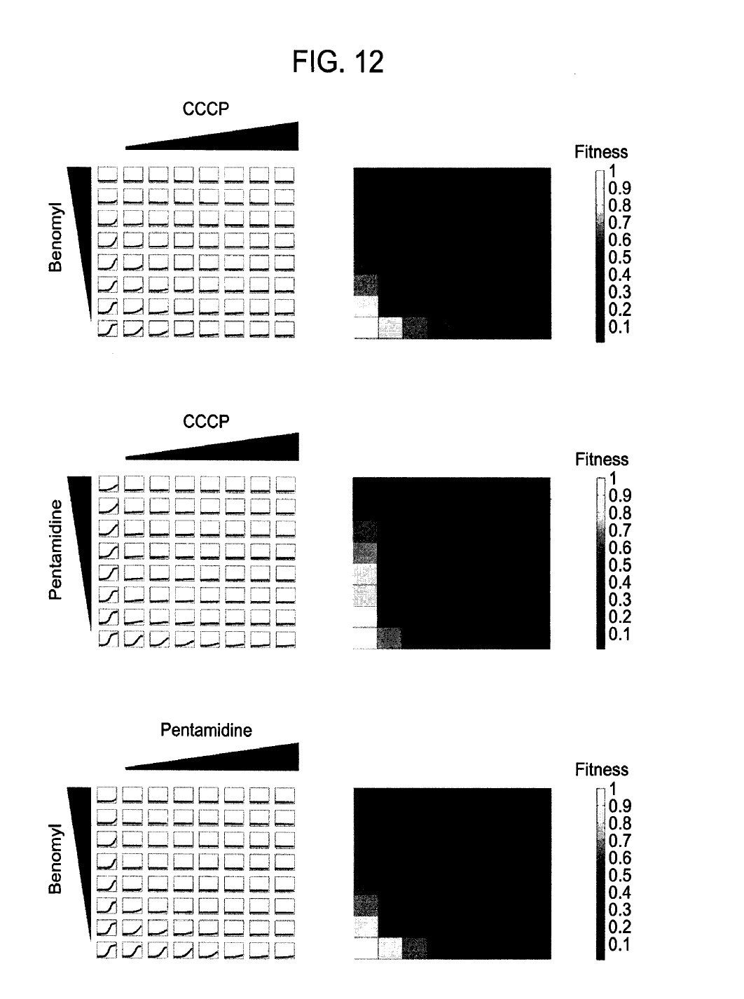

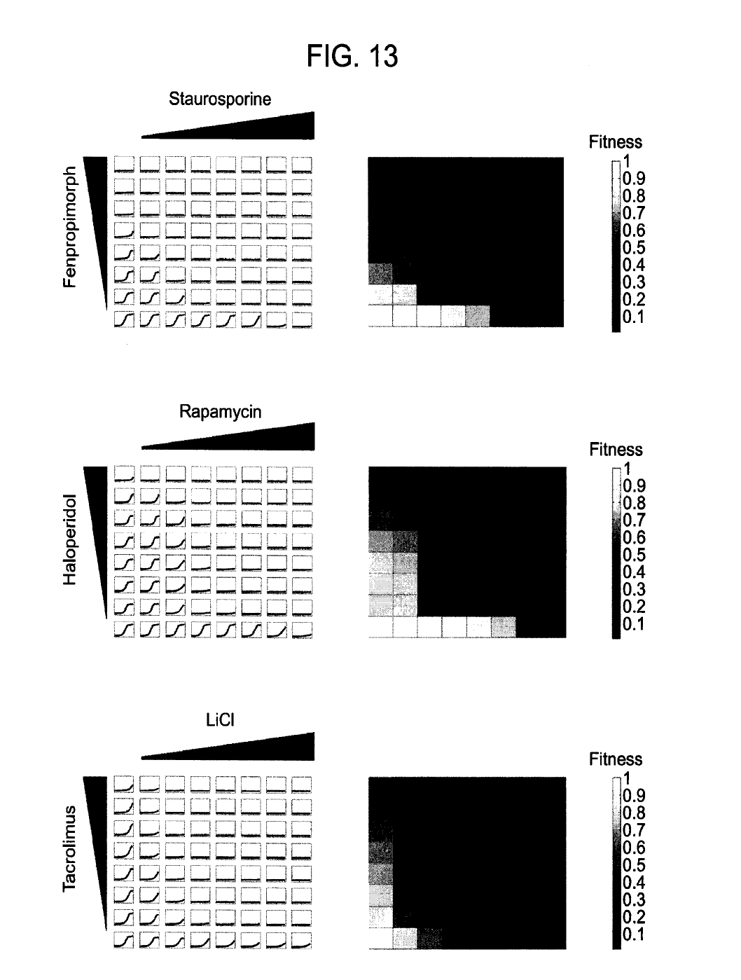

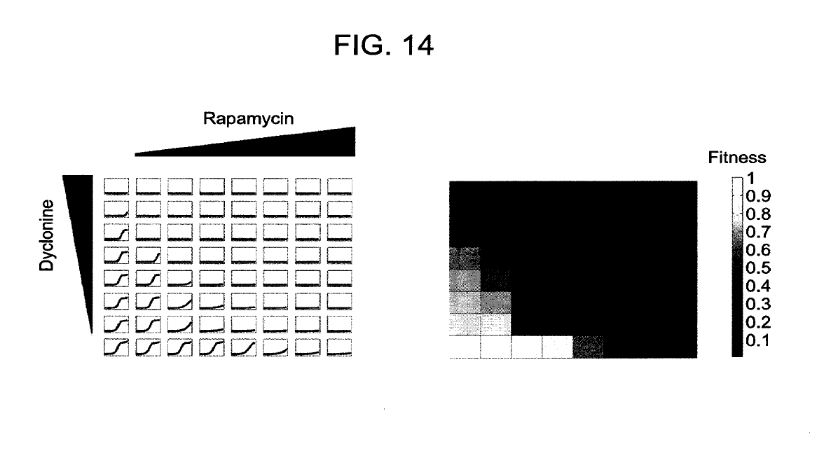

Left panels of FIGS. 2-14 show time courses of S. cerevisiae growth for several combinations of the reported synergistic drug pairs. Right panels give a summary representation of growth curves. Fitness in a given experiment is defined by the area under the growth curve divided by the area under the growth curve with no drugs present. White, grey and black indicate normal, slow and no growth, respectively. With reference to FIG. 1b, when the cell growth inhibition shows a concave shape in the concentration matrix experiment, then we have considered drugs to be synergistic. All aforementioned synergistic agent pairs showed this pattern. For example, treatment with 7 μg/ml latrunculin B or 126 μM tacrolimus only mildly reduced yeast growth. However, a combination of 1 μg/ml latrunculin B and 18 μM tacrolimus completely blocked yeast growth. Therefore, latrunculin B and FK506 acted synergistically to reduce yeast growth rate. FIGS. 2-14 demonstrate various synergistic drug pairs. Note that these empirical measures of growth are measuring the rate of growth in the size of the population of S. cerevisiae cells, which captures both fungistatic and fungicidal effects. Reducing yeast growth rate is an important criterion for a therapeutic treatment for local or systemic yeast infections.

Experiments were conducted as follows: In every experiment, S. cerevisiae cells (strain BY4741) in 100 μl of rich media were supplemented with 2% glucose and 2% DMSO. A culture having an initial OD₅₉₅ (a measure of cell density) of ˜0.1 was grown at 30° C. for 24 hours while measuring the OD₅₉₅ every 15 minutes. The curves in the left panels represent the cell density change in time under a variety of drug concentrations and combinations. Drugs were suspended in DMSO at 100× concentration of the maximum concentration and this stock was used to make the desired final concentrations of each drug.

Other embodiments will be evident to those of skill in the art. It should be understood that the foregoing description is provided for clarity only and is merely exemplary. The spirit and scope of the present invention are not limited to the above examples, but are encompassed by the following claims. All publications and patent applications cited above are incorporated by reference herein in their entirety for all purposes to the same extent as if each individual publication or patent application were specifically indicated to be so incorporated by reference.

What is claimed is:

1. A method of treating a condition in an individual in need of such treatment comprising administering at least one combination of staurosporine & tacrolimus; latrunculin B & tacrolimus; terbinafine & tacrolimus; fenpropimorph & tacrolimus; haloperidol & tacrolimus; tacrolimus and anisomycin; tacrolimus & cantharidin; tacrolimus & cycloheximide; tacrolimus & radicicol; terbinafine & staurosporine; dyclonine & staurosporine; haloperidol & staurosporine; terbinafine & latrunculin B; fenpropimorph & latrunculin B; dyclonine & latrunculin B; haloperidol & latrunculin B; terbinafine & rapamycin; terbinafine & calyculin A; fenpropimorph & terbinafine; dyclonine & terbinafine; haloperidol & terbinafine; terbinafine & 5-fluorouracil; terbinafine & anisomycin; terbinafine & carbonyl cyanide 3-chlorophenylhydrazone; terbinafine & cantharidin; terbinafine & radicicol; fenpropimorph & rapamycin; fenpropimorph & calyculin A; dyclonine & calyculin A; haloperidol & calyculin A; benomyl & carbonyl cyanide 3-chlorophenylhydrazone; pentamidine & carbonyl cyanide 3-chlorophenylhydrazone; benomyl & pentamidine; fenpropimorph & staurosporine; haloperidol & rapamycin; tacrolimus & lithium; or dyclonine & rapamycin in a manner to produce a synergistic effect within the individual sufficient to treat the individual.

2. (canceled)

3. (canceled)

4. A combination comprising tacrolimus and terbinafine, each of tacrolimus or terbinafine present in either a free form or in the form of a pharmaceutically acceptable salt and optionally at least one pharmaceutically acceptable carrier.

5. A combination comprising tacrolimus and fenpropimorph, each of tacrolimus or fenpropimorph present in either a free form or in the form of a pharmaceutically acceptable salt and optionally at least one pharmaceutically acceptable carrier.

6. A combination comprising tacrolimus and haloperidol, each of tacrolimus or haloperidol present in either a free form or in the form of a pharmaceutically acceptable salt and optionally at least one pharmaceutically acceptable carrier.

7. A combination comprising tacrolimus and anisomycin, each of tacrolimus or anisomycin present in either a free form or in the form of a pharmaceutically acceptable salt and optionally at least one pharmaceutically acceptable carrier.

8. A combination comprising tacrolimus and cantharidin, each of tacrolimus or cantharidin present in either a free form or in the form of a pharmaceutically acceptable salt and optionally at least one pharmaceutically acceptable carrier.

9. (canceled)

10. (canceled)

11. (canceled)

12. (canceled)

13. (canceled)

14. (canceled)

15. (canceled)

16. (canceled)

17. (canceled)

18. A combination comprising terbinafine and rapamycin, each of terbinafine or rapamycin present in either a free form or in the form of a pharmaceutically acceptable salt and optionally at least one pharmaceutically acceptable carrier.

19. A combination comprising terbinafine and calyculin A, each of terbinafine or calyculin A present in either a free form or in the form of a pharmaceutically acceptable salt and optionally at least one pharmaceutically acceptable carrier.

20. A combination comprising terbinafine and fenpropimorph, each of terbinafine or fenpropimorph present in either a free form or in the form of a pharmaceutically acceptable salt and optionally at least one pharmaceutically acceptable carrier.

21. A combination comprising terbinafine and dyclonine, each of terbinafine or dyclonine present in either a free form or in the form of a pharmaceutically acceptable salt and optionally at least one pharmaceutically acceptable carrier.

22. A combination comprising terbinafine and haloperidol, each of terbinafine or haloperidol present in either a free form or in the form of a pharmaceutically acceptable salt and optionally at least one pharmaceutically acceptable carrier.

23. (canceled)

24. A combination comprising terbinafine and anisomycin, each of terbinafine or anisomycin present in either a free form or in the form of a pharmaceutically acceptable salt and optionally at least one pharmaceutically acceptable carrier.

25. (canceled)

26. A combination comprising terbinafine and cantharidin, each of terbinafine or cantharidin present in either a free form or in the form of a pharmaceutically acceptable salt and optionally at least one pharmaceutically acceptable carrier.

27. (canceled)

28. (canceled)

29. A combination comprising fenpropimorph and calyculin A, each of fenpropimorph or calyculin A present in either a free form or in the form of a pharmaceutically acceptable salt and optionally at least one pharmaceutically acceptable carrier.

30. A combination comprising dyclonine and calyculin A, each of dyclonine or calyculin A present in either a free form or in the form of a pharmaceutically acceptable salt and optionally at least one pharmaceutically acceptable carrier.

31. A combination comprising haloperidol and calyculin A, each of haloperidol or calyculin A present in either a free form or in the form of a pharmaceutically acceptable salt and optionally at least one pharmaceutically acceptable carrier.

32. (canceled)

33. (canceled)

34. A combination comprising pentamidine and benomyl, each of pentamidine or benomyl present in either a free form or in the form of a pharmaceutically acceptable salt and optionally at least one pharmaceutically acceptable carrier.

35. (canceled)

36. (canceled)

37. (canceled)

38. A combination comprising dyclonine and rapamycin, each of dyclonine or rapamycin present in either a free form or in the form of a pharmaceutically acceptable salt and optionally at least one pharmaceutically acceptable carrier.

39. A kit containing a pharmaceutical composition comprising: at least one of a combination of staurosporine & tacrolimus; latrunculin B & tacrolimus; terbinafine & tacrolimus; fenpropimorph & tacrolimus; haloperidol & tacrolimus; tacrolimus and anisomycin; tacrolimus & cantharidin; tacrolimus & cycloheximide; tacrolimus & radicicol; terbinafine & staurosporine; dyclonine & staurosporine; haloperidol & staurosporine; terbinafine & latrunculin B; fenpropimorph & latrunculin B; dyclonine & latrunculin B; haloperidol & latrunculin B; terbinafine & rapamycin; terbinafine & calyculin A; fenpropimorph & terbinafine; dyclonine & terbinafine; haloperidol & terbinafine; terbinafine & 5-fluorouracil; terbinafine & anisomycin; terbinafine & carbonyl cyanide 3-chlorophenylhydrazone; terbinafine & cantharidin; terbinafine & radicicol; fenpropimorph & rapamycin; fenpropimorph & calyculin A; dyclonine & calyculin A; haloperidol & calyculin A; benomyl & carbonyl cyanide 3-chlorophenylhydrazone; pentamidine & carbonyl cyanide 3-chlorophenylhydrazone; benomyl & pentamidine; fenpropimorph & staurosporine; haloperidol & rapamycin; tacrolimus & lithium; dyclonine & rapamycin; a pharmaceutically acceptable carrier and instructions for use.
